(19) United States
(12) Patent Application Publication
(10) Pub. No.: US 2003/0143572 A1
(43) Pub. Date: Jul. 31, 2003

(54) Molecular diagnostic and computerized decision support system for selecting the optimum treatment for human cancer

(76) Inventors: Mou-Ying Fu Lu, Lake Bluff, IL (US); Rong Yu, Pearland, TX (US)

Correspondence Address: GREENBERG TRAURIG, P.C., 77 WEST WACKER DRIVE, CHICAGO, IL, 60601-1732, US

(21) Appl. No.: 10/216,509

(22) Filed: Aug. 9, 2002

Related U.S. Application Data
(60) Provisional application No. 60/312,005, filed on Aug. 13, 2001.

(51) Int. Cl. C12Q 1/68; C12P 19/34

(52) U.S. Cl. 435/6; 435/91.2

(57) ABSTRACT

A computerized decision support system and method for predicting which of one or more drugs suitable to treat a cancerous condition in a patient are the optimum drug(s), where such selection is based upon the particular patient's genotype. A PCR kit and/or a gene chip detects multiple genes, expressions and/or mutations associated with a particular cancer using a sample of the patient's tissue or blood. A detector accepts the gene chip and analyzes the patient's genotype; and a computerized system using a database which associates patient genotypes and the efficacy and toxicity of various anti-cancer drugs used in treating patients with a particular cancerous condition connected to the detector correlates the output of the detector to the database to provide a recommendation as to which drugs are optimum for treating the patient's cancer.

### BACKGROUND OF THE INVENTION

1. Field of the Invention

The present invention relates to the treatment of humans suffering from disease, and in particular, a computerized decision support system and method for predicting which of one or more drugs suitable to treat a cancerous condition in a patient are the optimum drug(s), where such selection is based upon the particular patient's genotype.

2. Prior Art

It is well known in the medical community that many cancerous conditions suffered by human patients may be treated by a single drug or a combination of drugs. In certain circumstances a medical professional may simply elect to treat a particular patient with one or more drugs selected from the multiple available drugs developed to treat the particular cancerous condition where the selection is made by the physician based upon the best available clinical data. This data may in some cases merely comprise the reported results of clinical trials which suggests that a particular drug has been demonstrated by trial as being effective in treating cancer in a percentage of trial patients who received the drug. This decision making process is consistent with the physician's desire to treat the patient with a regiment of drugs by trial and error which have the best chance of curing the cancerous conditions.

It is further known that the effectiveness of a particular drug in treating a medical condition, and in particular a cancerous condition, is not necessarily consistent from patient to patient. A drug that works well to treat a condition in one patient may not work at all to treat the very same condition suffered by another patient. In some cases the effectiveness of a particular drug may range from 30 to 70 percent across a patient group. Moreover one or more specific groups of people within the overall population may not even be viable candidates for the drug. At the same time, a particular drug if used to treat a cancer may produce unwanted side effects in a patient or may even be toxic to that patient. As with the effectiveness of a drug, the side effects caused by a particular drug are known to also vary from patient to patient wherein one patient may experience few if any undesired side effects from receiving a particular drug while another patient may suffer a great number of side effects, some potential lethal.

Variability relating to both the efficacy and toxicity of a particular cancer drug among the given population is well known to the medical community. Indeed, it is known that patients with a particular genotype are likely to react similarly to particular given drug while patients with a different genotype may react differently to that very same drug. Accordingly, there exists a known database of information which documents these findings—developed typically through clinical trial. Indeed, as new drugs are developed and as existing drugs are used more and more, the database grows.

There does exist prior art systems which are designed to identify a particular gene as existing within a given patient towards determining the genotype of that patient. In certain prior art systems, a process is used to determine whether a particular patient possesses a particular genotype wherein such analysis is accomplished through the use of detector devices such as the FISH device manufactured and sold by Vysis/Abbott. In such a prior art system only one gene is measured and correlated to one drug. This system only presents the operator with an indication as to the degree to which the particular gene is amplified within the patient sample.

In the case of using PCR technique, there is no known prior art using the multiple genes for the determination of the effectiveness of the marketed cancer drugs.

The foregoing described prior art system unfortunately suffers from several potential shortcomings. In particular, such a prior art system may often operate ineffectively given that only one gene's measurement is detected—one drug at a time. In order to detect the presence of multiple gene types within a patient tissue sample that relates to multiple drugs, the process must be repeated again and again for each of the genes and drug combinations. In most cases these combinations are unknown.

Moreover, a potentially more significant shortcoming is the fact that the detector generates an output which has little if any direct meaning to the operator, be they a physician or technician. In practice, the physician or technician using such a prior art device must interpret the output generated by the detector to determine whether or not a particular drug among many typically used might be suitable for treating a given patient. In order to make such a determination, the operator must take the results of the one or more repeated analysis output by the detector and compare those results to the raw database of information assembled from the multiple clinical trials which have been conducted in the medical community summarizing the results of trials and which specifically indicates which drugs have been proven by trial as being useful in treating patients having a particular genotype. Furthermore, the drug's response to an individual's genotype data normally is not available or has not been established.

One further shortcoming of the foregoing prior art example is the fact that the system may be subject to error introduced by the need to repeat the process multiple times in order to identify whether or not genes/mutations do or do not exist or are up or down regulated within a patient's sample. It is entirely possible that a technician may contaminate or otherwise mishandle a single tissue sample of among a series of multiple tissue samples where that one defective sample if properly prepared would potentially have indicated and produced the most desirable result. Furthermore, there may not be sufficient tumor samples from a patient to conduct many tests. In such a case, the physician may proceed to prescribe a less than optimum drug for treating the patient all the while being completely unaware that one pass through the detector among the many used to reach the result was defective.

Accordingly, it is an object of the present invention to provide a system which can be used by doctors to identify which pharmaceutical drugs from among several potential choices is indeed the most appropriate to treat a patient's particular medical condition.

Specifically, it is desirable to determine which pharmaceutical will have the greatest effectiveness with the least potential for causing toxic reaction or other side effects based upon the patient's genotyping. For example, using the present system, a particular pharmaceutical can be identified as being optimum for treating a cancerous condition based upon the patient's genotyping where a different pharmaceutical would be identified for a patient having a different genotyping.

To address the potential ineffectiveness of a particular pharmaceutical, doctors may prescribe a combination of drugs to treat a given condition, such as breast cancer. Such combinations may lead to further or increased side effects. It is therefore desirable and it is an additional object of the present invention to identify which singular drugs or known combinations of drugs are most effective in treating a given condition.

It is further an object of the present invention to eliminate the trial and error prior art practice of prescribing anti-cancer drugs and to provide a physician with a diagnostic tool and system that, based upon the individual patient's genotyping, serves to predict the outcome of a particular pharmaceutical before treatment even begins.

It is an additional object of the present invention to identify which drugs are optimum for treating breast cancer in a patient having a particular genotype.

It is another object of the present invention to identify which drugs are optimum to treat other cancerous conditions in patients.

It is another object of the present invention to provide a computerized decision support system to provide in plain language to a physician a recommendation as to the optimum anti-cancer drug to prescribe for a patient.

These and other desirable characteristics of the invention will become apparent in light of the present specification, including claims, and drawings.

### SUMMARY OF THE INVENTION

The present invention discloses a computerized decision support system and apparatus for selecting the optimum treatment for a cancerous condition in a human patient. The system comprises a PCR kit and/or a gene chip designed to detect multiple genes, expressions and/or mutations associated with a particular cancer using a sample of the patient's tissue or blood; a detector for accepting receipt of the gene chip toward analyzing the patient's genotype; a database describing the correlation of patient genotypes and the efficacy and toxicity of various anti-cancer drugs used in treating patients with a particular cancerous condition; and a computerized decision support system operably connected to the detector for correlating the output of the detector to the database. The operator is thereby provided with a definitive recommendation as to which drug or drugs are deemed optimum for treating the patient's cancer;

A method for selecting the optimum treatment for a cancerous condition in a human patient, is also disclosed comprising the steps of isolating mRNA from a patient's tumor or blood sample with an extraction buffer; synthesizing and amplifying cDNA in a patient's tumor or blood sample with primers highly specific for targeted cancer genes; detecting cancer genes, mutations using a kit and/or a gene chip; analyzing and interpreting PCR and/or gene chip results using a detector linked to a computerized decisions support system running a diagnostic software program with accompanying database for providing an indication of the drug which is optimum for treating the patient's cancer with the least likely chance for a drug interaction.

In one embodiment of the present invention, the method the step of isolating mRNA from a patient's tumor or blood sample comprises the substeps of: homogenizing a sample of the patient's tumor, blood or serum in 1 ml of denaturing solution containing 4M guanidine thiocyanate, 25 mM sodium citrate, and 0.1 mM 2-mercaptoethanol; mixing the resultant and homogenizing sequentially with 0.1 ml of 49:1 chloroform/isoamyl alcohol; incubating the resulting mixture for 15 minutes on ice and centrifuging at 10,000×g for 20 minutes at 4 degrees C.; transferring the upper aqueous phase into a new container and mixing with 1 ml of 100% isopropanol; incubating the resulting mixture at −20 degrees C. for thirty minutes at 10,000×g for 10 minutes; washing the resulting pellet with 1 ml of 75% ethanol and redissolving in RNase-free water; and then quantifying the resulting RNA sample on a spectophotometer at 260 nm and stored at −70 degrees C.

The step of synthesizing and amplifying CDNA in a patient's tumor or blood sample with specific primers for breast cancer genes further comprises the substeps of adding the RNA sample (1 μg) into 25 μg of 2× reaction mix containing 0.4 mM of each dNTP, 2.4 mM MgSO4, 16 U reverse transcriptase, and 2.5 U Tag DNA polymerase, and 10 μM cDNA amplification primers for breast cancer genes; adjusting the final solution volume to 50 μl with autoclaved distilled water; performing cDNA synthesis and amplification using a DNA Thermal Cycler with the following programs,—cDNA synthesis performed at 1 cycle of 45-55 degrees C. for 20-30 minutes, followed by an incubation at 94 degrees C. for two minutes,—cDNA amplification performed at 35-40 cycles of 94 degrees C. for 15 s (Denature)/55-60 degrees C. for 30 s (Anneal)/68-72 degrees C. for 1 minute (Extend), and -final extension performed at 1 cycle of 72 degrees C. for 5-10 minutes.

As disclosed, the breast cancer genes amplified with primers consist of ER Alpha, Her2, ErbB1, BRCA1 and BRCA2.

In the preferred embodiment, the step of detecting and analyzing the PCR product further comprises the substeps of resolving the PCR product by electrophoresis in 1.5% agarose gel; visualizing by electrofluorescence; and analyzing the number of PCR fragments using a detector device linked to a computerized decision support system. This system automatically correlates the output of the detector to a database comprising the results of clinical studies testing the efficacy and toxicity of various drugs in treating patients with particular genotypes having breast cancer; and providing an indication of one or more drugs which is optimum for treating the patient's breast cancer with the most effective outcome and the least amount of side effect.

In such embodiment, the PCR primer pairs comprise: ERα 5′-gctactgtgcagtgtgcaat (F), 5′-tcgtatcccacctttcatca (B); Her2 5′-aggatatccaggaggtgcag (F), 5′-actgctcatggcagcagtca (B); ErbB1 5′-gtggagaactctgagtgcat (F), 5′-cgaggatttccttgttggct (B); BRCA2 5′-ctgtccaggtatcagatgct (F), 5′-atgtgtggcatgacttggca (B); and BRCA1 5′-tagctgatgtattggacgtt (F), 5′-gagatctttggggtcttcag (B).

Another embodiment of the invention includes an integrated detector/analyzer which is designed to combine the function of PCR and gene chip reader(s). The output from the integrated detector/analyzer is linked to a computerized decision support system.

### BRIEF DESCRIPTION OF THE DRAWINGS

FIG. 1 of the drawings is a schematic representation of the primary components of the present invention, consisting of a sample preparation buffer, PCR detection kit, SNP gene chip and integrated analyzer;

FIG. 2 of the drawings is a schematic representation of the operation of the present invention, including preparing the blood or tissue sample, performing PCR reaction and gene chip hybridization, and detecting and analyzing the results;

FIG. 3 of the drawings is a schematic representation of the output of five breast cancer genes amplified on a PCR machine with the specific primers; and

FIG. 4 of the drawings is a schematic representation of the clinical decision support software used to assist physicians to prescribe the most effective available drugs.

### DETAILED DESCRIPTION OF THE DRAWINGS

While this invention is susceptible of embodiment in many different forms, there is shown in the drawings and will be described herein in detail, a specific embodiment, with the understanding that the present invention is to be considered as an exemplification of the principles of the invention and is not intended to limit the invention to the embodiment illustrated.

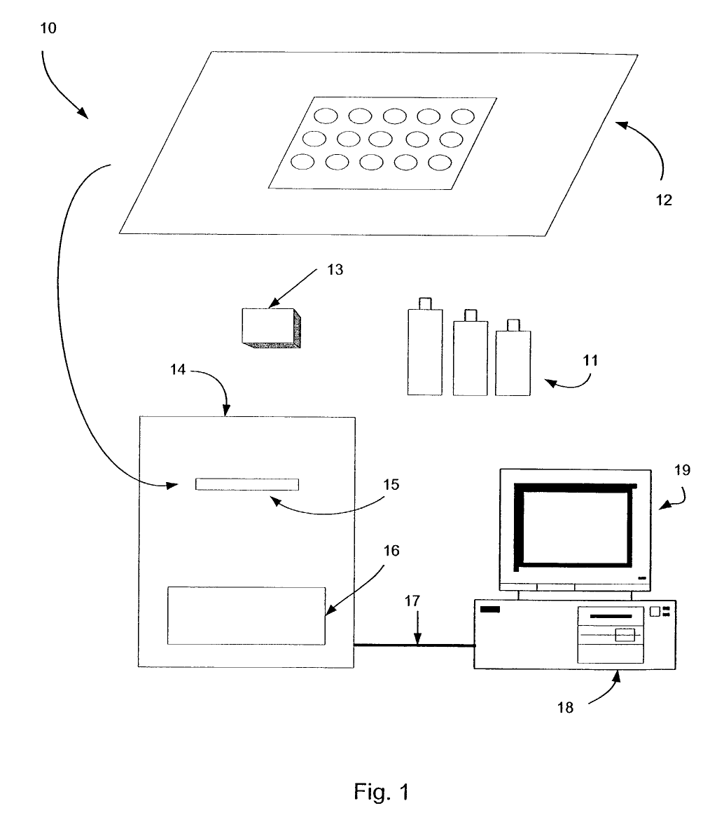

FIG. 1 of the drawings illustrates one embodiment of the present invention. The system for selecting the optimum treatment for a cancerous condition in human patient 10 is shown schematically as comprising chemicals and compounds 11 suitable for preparing the patient's blood or tissue sample, a gene chip 12 plated on a glass slide, a PCR kit 13 containing specific primers and reagents for detection of breast cancer genes, and a detector 14 and computer 17 running a bioinformatic software program.

A patient's blood or tumor tissue sample is prepared and hybridized with gene chip 12 or amplified with PCR detection kit 13, and then input into receptacle 16 of detector 14. The analysis of the gene chip 12 or PCR reaction is performed by detector 14 and is interpreted by a bioinformatic software package 17 running on computer 18. The output is displayed on monitor 19 that presents the results of the analysis to the doctor in plain language.

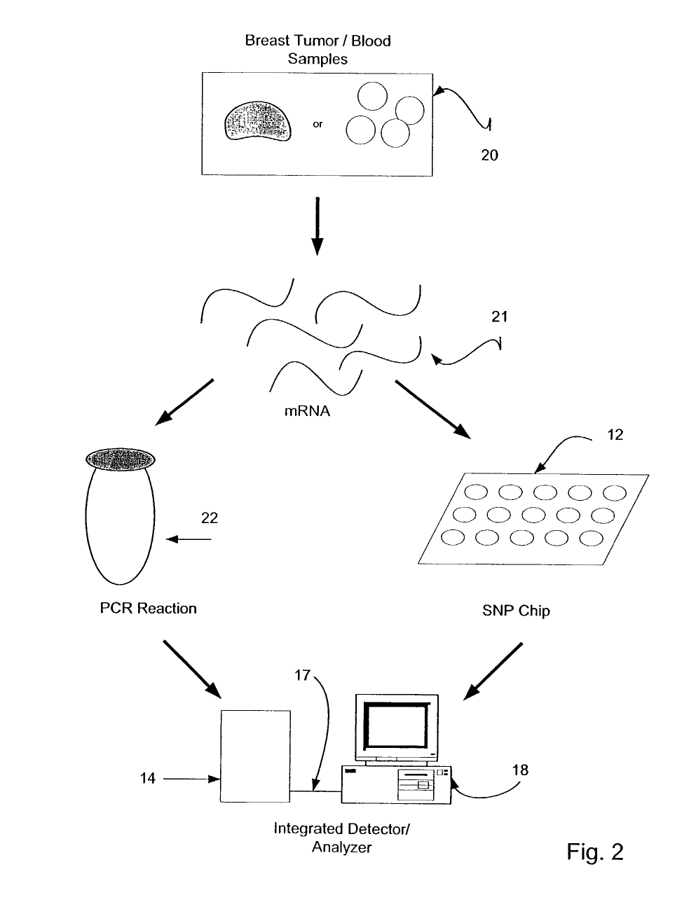

FIG. 2 of the drawings illustrates the genomic technology used in the present invention. High purity of mRNA 21 is prepared from the patient's blood or tumor tissue 20 with a unified extraction buffer as described by the following example. The tumor tissues or blood cells from a patient are homogenized in 1 ml of denaturing solution containing 4M guanidine thiocyanate, 25 mM sodium citrate, and 0.1 mM 2-mercaptoethanol. The momogiate is mixed sequentially with 0.1 ml of 49:1 chloroform/isoamyl alcohol. The resulting mixture is incubated for 15 minutes on ice and centrifuged at 10,000×g for 20 minutes at 4 ° C. The upper aqueous phase is transferred into a new tube and mixed with 1 ml of 100% isopropanol. The mixture is incubated at −20 ° C. for 30 minutes and centrifuged at 10,000×g for 10 minutes. The resulting pellet is washed with 1 ml of 75% ethanol and redissolved in RNase-free water. The RNA sample 21 is quantified on a spectophotometer at 260 nm and used for the detection of expression or mutation of cancer genes with PCR kit or SNP chip, respectively. The preparation of the blood or tumor tissue samples may be performed manually or alternatively by an automated unit.

For detection of gene expression using PCR kit 22, cDNA is synthesized and amplified in one-step. The RNA sample (1 μg) is added into 25 μg of 2× reaction mix containing 0.4 mM of each dNTP, 2.4 mM MgSO4, 16 U reverse transcriptase, and 2.5 U Tag DNA polymerase, and 10 μM cDNA amplification primers for breast cancer genes, ER Alpha, Her2, ErbB1, BRCA1 and BRCA2. The final reaction volume is adjusted to 50 μl with autoclaved distilled water. cDNA synthesis and amplification are performed using a DNA Thermal Cycler with the following programs.

A) cDNA synthesis: perform 1 cycle of 45-55° C. for 20-30 minutes, followed by an incubation at 94° C. for 2 minutes.

B) cDNA amplification: perform 35-40 cycles of 94° C. for 15 s (Denature)/55-60° C. for 30 s (Anneal)/68-72° C. for 1 minute (Extend).

C) Final extension: perform 1 cycle of 72° C. for 5-10 minutes.

The resulting reaction is analyzed with a detector which separates the DNA fragments into different groups according to the size and determines the number of copy in each group.

For detection of gene mutation 12, a pre-fabricated disease specific (such as breast cancer, liver cancer or ovarian cancer) SNP gene chip is provided and consists of chemically treated DNA fragments spotted on a plate. These DNA fragments are designed specifically for the detection of gene site mutation related to different cancer development stages and drug response. The preparation of the plate may be significantly different based on commercially available products. A unique technology of the present disclosure relates to specifying the content or what DNAs and/or their fragments are placed on the gene chip.

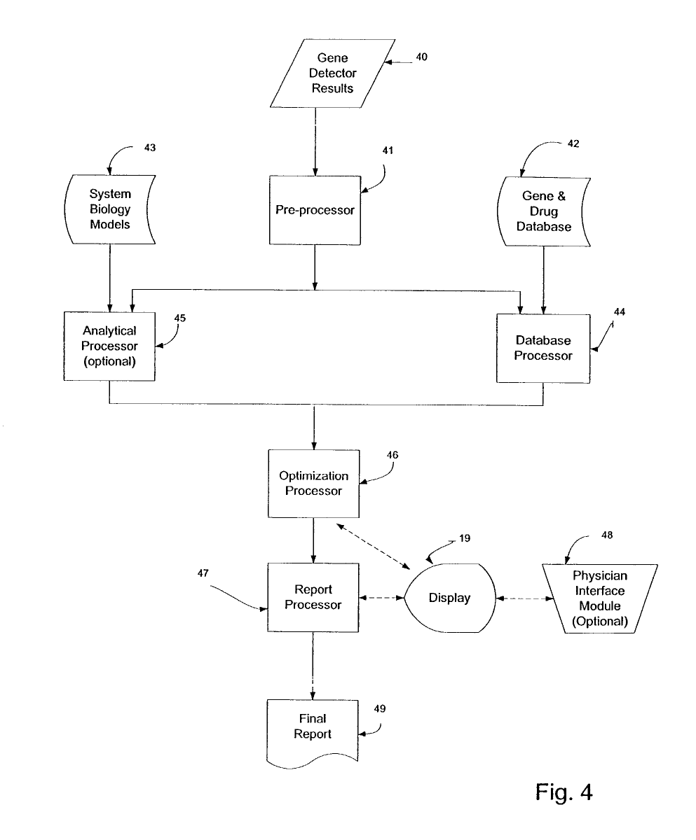

The purified mRNA sample 21 is labeled by direct incorporation of fluorescent Cy3-dUTP (red color) or Cy5 dUTP (green color) in reverse transcription. After labeling, the sample is hybridized with the oligonucleotides plated on gene chip in a automate hybridization chamber. The chip is then processed into an integrated detector 14 and analyzed for fluorescence intensity which is further converted to the gene mutation pattern and its relevance to drug response using GDC Clinical Decision Support Software (CDSS) 17 as detailed in FIG. 4. CDSS is running on computer 18. The output is displayed on monitor 19 that presents the results of the analysis to the doctor in plain language.

One unique aspect of the present invention is to define patient's genotype by measuring both gene expression and mutation in a combined procedure, and to convert these data to the most appropriate drug therapy.

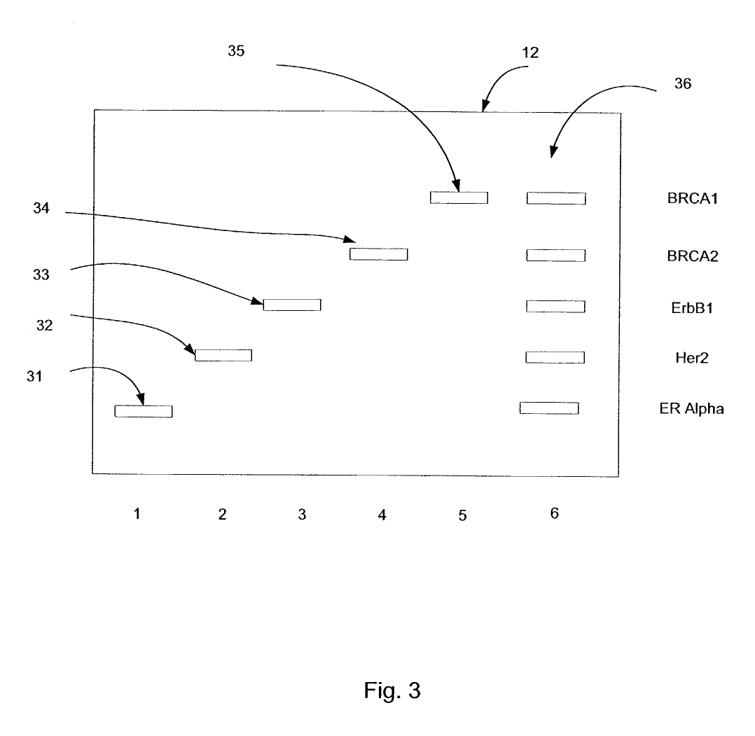

The FIG. 3 illustrates the specificity of PCR primers used for the detection of breast cancer genes ERα, Her2, ErbB1, BRCA1 and BRCA2 by gel electrofluorescence. As illustrated in lanes 1-5, numbered 31, 32, 33, 34 and 35, each contain PCR reactions using individual gene primer pairs. Lane 6 , numbered 36, as illustrated contains PCR reaction using a mixture of all 5 gene primer pairs. The mRNA sample used for PCR reactions is isolated from MCF7, a breast cancer cell line.

In FIG. 2 the detector 14 used in the preferred embodiment has the key components which include more than one sensor, such as immunohistochemistry, fluorescent etc., an interface chip linking the biological genotyping, interface circuit board connecting the detector 14 to computer 18 running a data and bioinformatic software package, a gene chip reader and holder and sample holder(s).

The detector 14 is equipped with an interface board (not shown) which serves to electronically connect detector 14 to personal computer 18 which runs a bioinformatic software program.

A bioinformatic software package is provided consisting of the correlation, calculation, criterion, and interpretation features which serve to correlate genetic data output from the detector 14 with a database of data toward providing the physician with a recommendation into plain English in order to assist doctors to select the most effective medicine with the least amount of side effect for patients. The interaction or correlation between individual genotyping and medicines is developed from clinical and/or published peer reviewed publications. This software may be further customized for a single disease or multiple diseases.

FIG. 4 of the drawings illustrates a flow chart which further describes the bioinformatic software program. With the increasing technological ability in providing patient diagnostics information at the molecular biology level and the increasing numbers of available patient treatment drugs in the market, a physician can no longer depend on the conventional method to prescribe an anti-cancer drug, which is basically a mere trial and error approach to prescribing a drug for a patient. The clinical decision support system and bioinformatic software of the present invention is designed to aid the physician in making decisions based on the available and affordable information regarding patient diagnosis, a clinical knowledge database, analytical biology models and physicians' empirical experience.

The schematic of FIG. 4 illustrates the process and components of the software used to provide the physician with the plain language recommendation as to which drugs to use for a particular patient. Step 40 illustrates data output from the detector 14 in terms of gene expression level and gene mutation type. This data output is supplied to a pre-processor 41 which is a module which maps the gene detector results into an algorithm that can be processed with system biology models and gene and drug database, 43 and 42. Gene and drug database is a module which stores the statistical association tables based on public domain or privately conducted clinical trial results. The basic data variables consist of patient genotype and patient drug responses determined over time. The system biology model 43 is the module that stores the multiple genes and multiple drug pathway analytical models at a molecular biology level based on public domain or privately conducted research. This module also stores disease development analytical process at cellular biology and molecular biology level.

Optimization processor 46 consists of a number of search algorithms that find the best fit results for the patient using the knowledge contained in the system biology models and gene and drug database or even physician's feedback,if desirable. Report processor 47 provides the computer analysis from the optimization processor 46 in a printout form 49 or on a computer screen 19.

Physician interface module 48 provides a physician an opportunity to do ‘what-if’ analysis using the optimization processor 46 based on the physician's empirical experience with his or her medical practices and the patient. Computer 18 and monitor 19 present recommendations as to the optimum drugs based upon a patient genotype to the doctor in an understandable manner, for example, listing the benefits of the drug, the efficacy for the patient's particular genotype, the drug's side effects based upon the patient's genotype and other relevant information.

### DETAILED DESCRIPTION OF THE OPERATION OF THE PRESENT SYSTEM AND METHOD

In the first step, the system isolates mRNA from patient tumor for blood sample with an improved extraction buffer. In the second step, the system synthesizes and amplifies cDNA in with highly specific primers for five breast cancer genes. Lastly, the system detects and analyzes PCR product with a detector apparatus linked to a PC running a diagnostic software program with accompanying database for prediction of gene and drug interaction.

In the embodiment of the present invention directed to selecting drugs to treat breast cancer, the PCR Detection of breast cancer genes is accomplished using the following specific gene primer pairs referred to above.

| Gene | Primer | Predicted Size |
|---|---|---|
| ERα | 5′-gctactgtgcagtgtgcaat (F) | 202 bp |
| | 5′-tcgtatcccacctttcatca (B) | |
| Her2 | 5′-aggatatccaggaggtgcag (F) | 416 bp |
| | 5′-actgctcatggcagcagtca (B) | |
| ErbB1 | 5′-gtggagaactctgagtgcat (F) | 603 bp |
| | 5′-cgaggatttccttgttggct (B) | |
| BRCA2 | 5′-ctgtccaggtatcagatgct (F) | 799 bp |
| | 5′-atgtgtggcatgacttggca (B) | |
| BRCA1 | 5′-tagctgatgtattggacgtt (F) | 1024 bp |
| | 5′-gagatctttggggtcttcag (B) | |

A patient's breast tumor sample and/or blood sample is prepared using a test kit according to the present invention, depending upon the assay or detector mode of the sensor used in the detector module. A patient's breast tumor sample is prepared using a one step detection method designed to detect multiple cancer genes to extract the predetermined mRMAs or genes related to the targeted drugs such as Herception, Tamoxifen and Fermera.

The prepared test sample with the selected mRMAs is applied to a gene chip or slide. The chip or slide is then placed in the detector sample holder which is, in turn, inserted into the detector apparatus. A bioinformatic software program serves to correlate and calculate the raw signals/data provided by the detector apparatus and will interpret the raw signals/data according to criteria and drug information stored in the system database. The bioinformatic software serves to translate genetic and drug data into plain spoken language (be it in English, Chinese, etc.) in order to assist doctors to select the most effective drug for treating the particular patient's breast cancer.

A sample of the raw signal or data generated by the system detector is as follows:

| Gene Type | Expression Level |
|---|---|
| ErbB2 | Up regulated (1.5) |
| ErbB1 | Up regulated (1.6) |
| Er Alpha | Down regulated (0.8) |
| BRCA1 | Up regulated (2.0) |
| BRCA2 | Down regulated (0.5) |

As illustrated, the foregoing raw data typically generated by the conventional detector unit is not particularly intuitive and does not readily convey to the physician or technician any direct indication of the drug most appropriate for treating the patient. Accordingly, the present invention preferably provides an output to the user which may consist of a plain language message which reads:

“Recommendation: May use Fermera and/or Herceptin. Tamoxifen may have resistant if BCL-2 gene is up regulated (use additional kit guide for BCL-2 testing)”.

A further example of the raw signal or data output by the system detector may be:

| Mutation Type | Expression Level |
|---|---|
| Val335Leu | Up regulated |
| Glu386Ter | Up regulated |

The system according to the present invention preferably generates an output to the user which reads:

“Recommendation: May not use 5-FU due to DPD protein related toxicity.”

To that end, the detection and analysis of PCR Products is accomplished whereby the PCR products are resolved by electrophoresis. The number of PCR fragments are analyzed with the detector 14 equipped with a fluorescent sensor and which is electronically linked 17 to the system PC.

It is a further aspect of the present invention to use the present method and apparatus to predict or identify the optimum drug for treating cancers other then breast cancer. Indeed, the present automated system can be used to identify an optimum drug for treating virtually any disease for which there exists an established correlation between a patient genotype and the efficacy and toxicity of each of a group of drugs developed to treat the general condition.

Accordingly, the foregoing description and drawings merely explain and illustrate the invention, and the invention is not limited except insofar as the appended claims are so limited, as those skilled in the art who have the disclosure before them will be able to make modifications and variations therein without departing from the scope of the invention.

We claim:

1. A computerized decision support system and apparatus for selecting the optimum treatment for a cancerous condition in a human patient, the apparatus comprising:
a PCR kit and/or a gene chip designed to detect, expressions and/or mutations of multiple genes associated with a particular cancer, using patient's tissue or blood samples;
an integrated detector for analyzing both PCR and gene chip results.
a detector for accepting receipt of the gene chip toward analyzing the patient's genotype;
a database describing the correlation of patient genotypes and the efficacy and toxicity of various anti-cancer drugs used in treating patients with a particular cancerous condition; and
a computerized decision support system operably connected to the detector for correlating the output of the detector to the database;
whereby the operator is provided with a definitive recommendation as to which drug or drugs are deemed optimum for treating the patient's cancer;

2. A method for selecting the optimum treatment for a cancerous condition in a human patient, the method comprising:
preparing PCR kit and gene chip;
isolating mRNA from a patient's tumor or blood sample with an extraction buffer;
synthesizing and amplifying cDNA in a patient's tumor or blood sample with primers highly specific for targeted cancer genes;
detecting cancer genes, mutations using a gene chip;
analyzing and interpreting PCR and/or gene chip results using a detector linked to a computerized decisions support system running a diagnostic software program with accompanying database for providing an indication of the drug which is optimum for treating the patient's cancer with the least likely chance for a drug side effect.

3. The method according to claim 2, wherein the step of isolating mRNA from a patient's tumor or blood sample comprises the substeps of:
homogenizing a sample of the patient's tumor, blood or serum in 1 ml of denaturing solution containing 4M guanidine thiocyanate, 25 mM sodium citrate, and 0.1 mM 2-mercaptoethanol;
mixing the resultant and homogenizing sequentially with 0.1 ml of 49:1 chloroform/isoamyl alcohol;
incubating the resulting mixture for 15 minutes on ice and centrifuging at 10,000×g for 20 minutes at 4 degrees C.;
transferring the upper aqueous phase into a new container and mixing with 1 ml of 100% isopropanol;
incubating the resulting mixture at −20 degrees C. for thirty minutes at 10,000×g for 10 minutes;
washing the resulting pellet with 1 ml of 75% ethanol and redissolving in RNase-free water; and
quantifying the resulting RNA sample on a spectophotometer at 260 nm and stored at −70 degrees C.

4. The method according to claim 2, wherein the step of synthesizing and amplifying cDNA in a patient's tumor or blood sample with specific primers for breast cancer genes further comprises the substeps of:
adding the RNA sample (1 μg) into 25 μg of 2× reaction mix containing 0.4 mM of each dNTP, 2.4 mM MgSO4, 16 U reverse transcriptase, and 2.5 U Tag DNA polymerase, and 10 μM cDNA amplification primers for breast cancer genes;
adjusting the final solution volume to 50 μl with autoclaved distilled water;
performing cDNA synthesis and amplification using a DNA Thermal Cycler with the following programs,
cDNA synthesis performed at 1 cycle of 45-55 degrees C. for 20-30 minutes, followed by an incubation at 94 degrees C. for two minutes;
cDNA amplification performed at 35-40 cycles of 94 degrees C. for 15 s (Denature)/55-60 degrees C. for 30 s (Anneal)/68-72 degrees C. for 1 minute (Extend); and
Final extension performed at 1 cycle of 72 degrees C. for 5-10 minutes.

5. The method according to claim 4 comprises the specific primers for breast cancer genes, ER Alpha, Her2, ErbB1, BRCA1 and BRCA2.

6. The method according to claim 2 for detecting and analyzing the PCR product further comprises the substeps of:
resolving the PCR product by electrophoresis in 1.5% agarose gel;
visualizing by electrofluores,ence; and
analyzing the number of PCR fragments using a detector device linked to a computerized decision support system.

7. A method using a clinical computerized decision support system for selecting the optimum treatment for a patient suffering from breast cancer, the method comprising:
combining a gene chip with PCR primers for the detection of particular breast cancer genes in a sample of the patient's tissue or blood;
optically inspecting the resulting chemical and biological reaction using a automated detector;
correlating using the software output from the integrated detector with a disease analytical models database and/or a database comprising the results of clinical studies testing the efficacy and toxicity of various drugs in treating patients with particular genotypes having breast cancer; and
providing an indication of one or more drugs which is optimum for treating the patient's breast cancer with the most effective outcome and the least amount of side effect.

8. The method according to claim 7, wherein the PCR primers comprise:
ERα 5′-gctactgtgcagtgtgcaat (F), 5′-tcgtatcccacctttcatca (B);
Her2 5′-aggatatccaggaggtgcag (F), 5′-actgctcatggcagcagtca (B);
ErbB1 5′-gtggagaactctgagtgcat (F), 5′-cgaggatttccttgttggct (B);
BRCA2 5′-ctgtccaggtatcagatgct (F), 5′-atgtgtggcatgacttggca (B); and
BRCA1 5′-tagctgatgtattggacgtt (F), 5′-gagatctttggggtcltcag (B).
